(19) United States
(12) Patent Application Publication
(10) Pub. No.: US 2025/0057529 A1
(43) Pub. Date: Feb. 20, 2025

(54) MEDICAL DEVICES AND RELATED METHODS FOR STEERING, ACTUATING, AND/OR CONTROLLING AN END EFFECTOR

(71) Applicants: Boston Scientific Scimed, Inc., Maple Grove, MN (US); Boston Scientific Medical Device Limited, Galway (IE)

(72) Inventors: Deepak Kumar SHARMA, Muzaffarnagar (IN); Charles A. GIBSON, Malden, MA (US)

(73) Assignees: Boston Scientific Scimed, Inc., Maple Grove, MN (US); Boston Scientific Medical Device Limited, Galway (IE)

(21) Appl. No.: 18/806,254

(22) Filed: Aug. 15, 2024

Related U.S. Application Data
(60) Provisional application No. 63/519,861, filed on Aug. 16, 2023.

(51) Int. Cl. A61B 17/072; A61B 17/00; A61B 17/29

(52) U.S. Cl. CPC A61B 17/072; A61B 2017/00398; A61B 2017/0046; A61B 2017/2927

(57) ABSTRACT

A medical device includes a handle, a shaft, an end effector, and a wire. The handle includes a rotatable knob and a longitudinally movable spool. The shaft extends from a distal end of the handle. The end effector is coupled to a distal end of the shaft. The wire extends from the handle, through the shaft, and to the end effector. Rotation of the knob rotates the wire independent of the shaft to rotate the end effector. Distal movement of the spool moves the wire distally independent of the shaft and transitions the end effector between at least a first configuration and a second configuration. Proximal movement of the spool urges the wire proximally. Urging the wire proximally deflects a deflectable portion of the shaft in one or more directions.

### CROSS-REFERENCE TO RELATED APPLICATIONS

This application claims the benefit of priority under 35 U.S.C. § 119 from U.S. Provisional Application No. 63/519,861, filed Aug. 16, 2023, which is incorporated by reference herein in its entirety.

### TECHNICAL FIELD

The disclosure relates generally to devices and methods for steering, articulating, and/or controlling an end effector. More specifically, aspects of the disclosure pertain to devices and/or related methods for steering, articulating, and/or controlling an end effector with a single pull wire.

### BACKGROUND

Medical procedures, for example, such as electrosurgical dissection, often involve lifting tissue (e.g., with a grasper) and tissue resection (e.g., with a cautery knife or snare). In particular, such procedures may be carried out by delivering an insertion device into a subject's body through a surgical incision, or via a natural anatomical orifice (e.g., mouth, vagina, or rectum), and performing the procedure or operation at a treatment site with an auxiliary device inserted through the insertion device. The auxiliary device may be coupled to tissue and may be used to lift the tissue, for example, by applying a proximal force to the tissue. The auxiliary device may include a single wire (e.g., a pull wire) that is used to lift tissue. However, the size (e.g., lateral cross-section or diameter) of the auxiliary device may be limited, for example, by the size of a working channel of the insertion device. The auxiliary device may also include one or more other wires, cables, or other components, for example, for articulation. As such, the size of the pull wire for lifting the tissue may be limited, thus limiting the lifting strength, durability, etc. of the pull wire, which may increase the duration, necessary skill or technique, risk of breakage or other damage, or otherwise reduce the effectiveness/accuracy of the procedure. Therefore, a need exists for devices and/or methods for steering and/or articulating an end effector.

### SUMMARY

This disclosure includes medical devices and methods for steering, articulating, and/or controlling an end effector. Each of the aspects disclosed herein may include one or more of the features described in connection with any of the other disclosed aspects.

In one or more examples, a medical device may include a handle, a shaft, an end effector, and a wire. The handle may include a rotatable knob and a longitudinally movable spool. The shaft may extend from a distal end of the handle. The end effector is coupled to a distal end of the shaft. The wire may extend from the handle, through the shaft, and to the end effector. Rotation of the knob may rotate the wire independent of the shaft to rotate the end effector. Distal movement of the spool may move the wire distally independent of the shaft and may transition the end effector between at least a first configuration and a second configuration. Proximal movement of the spool may urge the wire proximally. Urging the wire proximally may deflect a deflectable portion of the shaft in one or more directions.

The medical device may include one or more of the following features. The shaft may include a deflectable portion that includes a plurality of openings arranged in one or more longitudinally extending rows. The deflectable portion may include four longitudinally extending rows of openings that are spaced circumferentially around the deflectable portion. A portion of the wire may include an intermediate section including a flat surface. The flat surface may at least partially overlap with at least one of the rows of openings. The flat surface may be configured to overlap with a respective row of the four longitudinally extending rows based on a rotational position of the wire in order to deflect the deflectable portion in four directions.

The medical device may further include a biasing element positioned within a portion of the handle to bias the distal movement of the spool. The medical device may further include a tube surrounding a proximal portion of the wire. The tube may include at least one flat outer surface to interact with an internal portion of the knob. The end effector may include two pivotable jaws that are movable between a closed configuration and an open configuration. The two pivotable jaws may be coupled to a distal end of the wire via respective end effector wires. A proximal end of the end effector may be coupled to a bushing, and the bushing may be rotatably coupled to a distal end of the shaft. A distal end of the wire may include a stepped diameter portion that is larger than a proximal portion of the wire. The handle may include a handle body with a slot that extends longitudinally through a portion of the handle body. A portion of the spool may be movable within the slot to control the longitudinal movement of the wire. The slot may include a narrow distal portion and a wide proximal portion. The handle may include one or more arms that partially surround the knob. A proximal end of the handle may include a ring.

In another aspect, a medical device may include a handle, a shaft, an end effector, and a wire. The handle may include a rotatable knob and a longitudinally movable spool. The shaft may extend from a distal end of the handle. The end effector may be rotatably coupled to a distal end of the shaft. The wire may extend from the handle, through the shaft, and to the end effector. A distal portion of the wire may include a distal extension and an intermediate section including a flat surface. Rotation of the knob may rotate the wire independent of the shaft to rotate the end effector. Distal movement of the spool may move the wire distally independent of the shaft and may transition the end effector between at least a first configuration and a second configuration.

The medical device may include one or more of the following features. The shaft may include a deflectable portion that includes four longitudinally extending rows of openings that are spaced circumferentially around the deflectable portion. The flat surface may at least partially overlap with at least one of the rows of openings, such that proximal movement of the spool urges the wire proximally. Urging the wire proximally may deflect the deflectable portion in one or more directions. The medical device may further include a tube surrounding a proximal portion of the wire. The tube may include at least one flat outer surface to interact with an internal portion of the knob.

In yet another aspect, a medical device may include a handle. The handle may include a first longitudinally movable actuator and a second longitudinally movable actuator. The medical device may further include a shaft extending from a distal end of the handle. The shaft may include a deflectable portion. The deflectable portion may include a plurality of openings arranged in a longitudinally extending row. The medical device may further include an end effector coupled to a distal end of the shaft. The medical device may further include a first wire extending from the handle, through the shaft, and to the end effector. The medical device may further include a second wire extending from the handle, through the shaft, and to the distal end of the shaft. Rotation of the shaft may rotate the end effector, the first wire and the second wire. Distal movement of the first actuator may move the first wire distally independent of the shaft and the second wire, and may transition the end effector between at least a first configuration and a second configuration. Distal movement of the second actuator may urge the second wire distally independent of the first wire. Urging the second wire distally may deflect the deflectable portion of the shaft in one or more directions.

The medical device may include one or more of the following features. The first wire may include a cylindrical distal section configured to couple to the end effector. The second wire may include an intermediate section including a flat surface. The flat surface may at least partially overlap with the longitudinally extending row.

Any of the examples described herein may have any of these features in any combination.

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts a perspective view of an exemplary medical device, including an enlarged view of a distal portion of the medical device.

FIG. 2A-2E depict various views of various aspects of the distal portion of the medical device of FIG. 1.

FIG. 3 is a partially cutaway view of a proximal portion of the medical device of FIG. 1.

FIG. 4A illustrates the proximal portion of the medical device of FIG. 1 undergoing a first manipulation, and FIG. 4B illustrates the distal portion of the medical device of FIG. 1 undergoing a corresponding first movement based on the first manipulation.

FIG. 5A illustrates the proximal portion of the medical device of FIG. 1 undergoing a second manipulation, and FIGS. 5B and 5C illustrate the distal portion of the medical device of FIG. 1 undergoing a corresponding second movement between two configurations based on the second manipulation.

FIGS. 6A-6C illustrate additional aspects of the distal portion of the medical device of FIG. 1.

FIG. 7 illustrates the distal portion of the medical device of FIG. 1 undergoing a longitudinal manipulation.

FIG. 8 illustrates the proximal portion of the medical device of FIG. 1 undergoing a rotational manipulation, and also illustrates the distal portion of the medical device of FIG. 1 undergoing a rotational movement based on the rotational manipulation of the proximal portion.

FIG. 9A illustrates another exemplary distal portion of a medical device.

FIG. 9B illustrates a proximal portion of the medical device depicted in FIG. 9A.

FIG. 10 illustrates a cross-sectional view of the distal portion of the medical device depicted in FIG. 9A.

FIG. 11 illustrates a deflectable portion of a shaft of the distal portion of the medical device of FIG. 9A.

FIGS. 12A and 12B illustrate the distal portion of the medical device of FIG. 9A in two different end effector configurations.

FIGS. 13A and 13B illustrate the distal portion of the medical device of FIG. 9A in two different shaft steering configurations.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

Embodiments of this disclosure seek to improve a user's ability to steer, articulate, and/or otherwise manipulate an end effector within a subject's body during a medical procedure, help reduce the need to remove and reintroduce an endoscope or other medical device into the subject's body, help treat tissue within the subject, reduce overall device size and/or cost, and/or reduce overall procedure time, among other aspects.

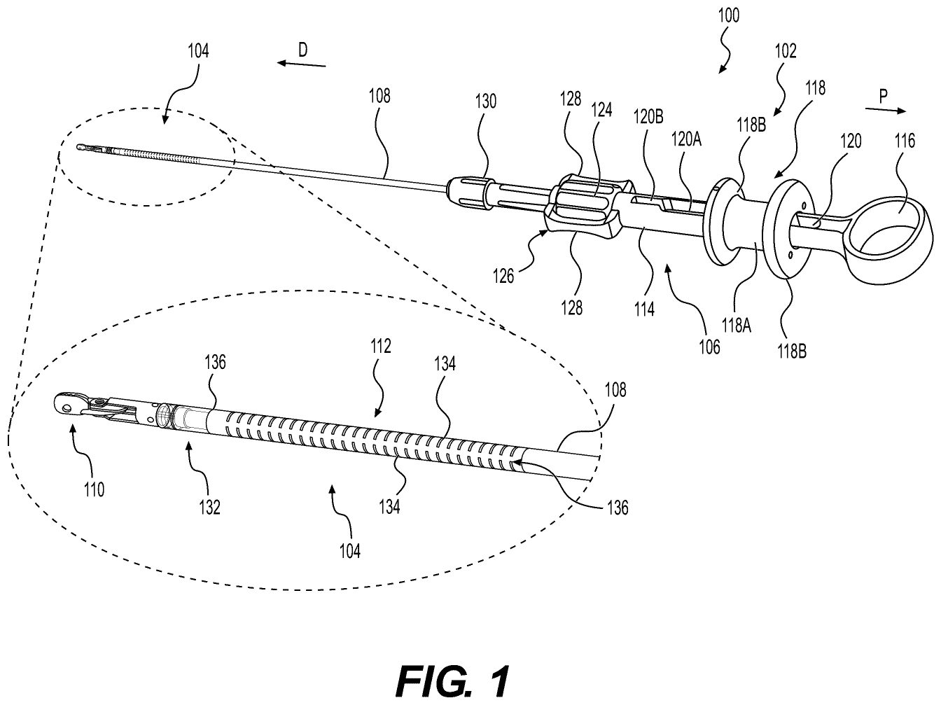

FIG. 1 illustrates a medical device 100, including a proximal portion 102 and a distal portion 104. FIG. 1 includes an enlarged view of distal portion 104. As shown in FIG. 1, medical device 100 includes a handle 106, for example, at proximal portion 102. Medical device 100 includes a shaft 108, for example, extending from a distal end of handle 106. Medical device 100 also includes an end effector 110 at a distal end of shaft 108, for example, at distal portion 104. A portion of shaft 108 also includes a deflectable portion 112, for example, at distal portion 104. As discussed in detail below, movement of various portions of handle 106 may allow for steering, articulating, and/or manipulating end effector 110, for example, in a plurality of degrees of freedom.

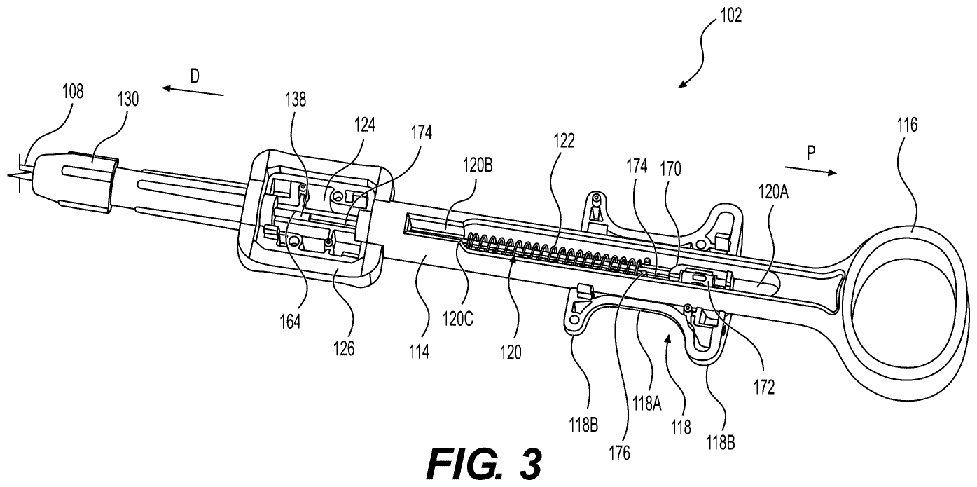

Handle 106 includes a main body 114, for example, including a ring 116 (e.g., a thumb ring), for example, at a proximal end of main body 114. Handle 106 also includes a first movable member or spool 118. Spool 118 may be an actuator and may include an indented portion 118A (e.g., with a relatively smaller lateral cross-section) and one or more (e.g., two) ridged or extended portions 118B (e.g., with a relatively larger lateral cross-section). In these aspects, indented portion 118A may receive one or more of the user's fingers, such that movement of the user's finger(s) controls the movement of spool 118. Main body 114 may include a slot 120, for example, extending longitudinally through a portion of main body 114, for example, from a position spaced distally from ring 116. A portion of spool 118 may extend into a portion of slot 120, such that spool 118 is movable (e.g., longitudinally movable distally and/or proximally) along slot 120. In these aspects, slot 120 may define a range of movement for spool 118. As discussed in detail below, spool 118 may be movable within slot 120, for example, proximally and/or distally, to control one or more aspects of end effector 110. Furthermore, slot 120 may include a wide portion 120A and a tapered or narrow portion 120B. As discussed in detail below, wide portion 120A may be proximal of narrow portion 120B, and the transition between wide portion 120A and narrow portion 120B may help to support or otherwise provide a stop surface for a biasing element 122 (FIG. 3, discussed below).

Additionally, handle 106 includes a second movable member or knob 124. For example, main body 114 may include a cage 126, for example, formed by two arms 128 that partially surround knob 124. Knob 124 and cage 126 may be distal of slot 120 on main body 114. As discussed in detail below, knob 124 may be an actuator and may be rotatable, for example, clockwise and/or counterclockwise, to control one or more aspects of end effector 110. For example, knob 124 may be rotatable about a longitudinal axis (e.g., a central longitudinal axis) of medical device 100.

Handle 106 may also include an end cap 130, for example, at a distal end of handle 106. End cap 130 may surround a proximal end of shaft 108. End cap 130 may help to couple shaft 108 to handle 106. In some aspects, end cap 130 may help to form a strain relief portion of medical device 100.

As mentioned, shaft 108 includes end effector 110 at a distal end. End effector 110 is coupled to shaft 108 at the distal end of shaft 108, for example, at a coupling portion 132. As discussed below, coupling portion 132 may help to allow for the movement, actuation, manipulation, or otherwise control of end effector 110. As shown, end effector 110 may be a forceps, for example, including two jaws. Nevertheless, this disclosure is not so limited, and end effector 110 may be any type of end effector, instrument, tool, or other device (e.g., grasper, snare, clip, stapler, ablation device, tome, suturing device, needle, knife, etc.).

Moreover, a distal portion of shaft 108 includes deflectable portion 112. Deflectable portion 112 may include a tube with a plurality of grooves, slits, or openings 134, for example, extending around respective portions of a circumference of deflectable portion 112. For example, deflectable portion 112 may include a plurality of openings 134. Each opening 134 of a group of openings may extend around approximately 75 degrees of the circumference of deflectable portion 112 of shaft 108. For example, if there are four openings 134 in a group of openings, each opening 134 may span approximately 60 to approximately 85 degrees, for example, approximately 75 degrees, of a circumference of deflectable portion 112. Additionally, groups of openings 134 may be spaced longitudinally from adjacent groups of openings 134 along a length of deflectable portion 112. In these aspects, deflectable portion 112 may include a plurality of longitudinally extending rows of openings 134. For example, if each group of openings 134 includes four openings 134, then deflectable portion 112 may include four longitudinally extending rows 136 of openings 134. As shown, each opening 134 in the rows 136 of openings 134 is spaced longitudinally from an adjacent opening 134 in the corresponding row. It is noted, however, that the size and spacing (longitudinal spacing, longitudinal width, circumferential spacing, circumferential length, etc.) of openings 134 and rows 136 may vary. In these aspects, openings 134 may be formed by laser cutting or otherwise cutting, for example, forming a laser cut pattern including rows 136 of openings 134. Additionally, although not shown, shaft 108 may include one or more outer layers, for example, radially surrounding deflectable portion 112. In these aspects, the one or more outer layers may be flexible, such that movement of deflectable portion 112 moves shaft 108.

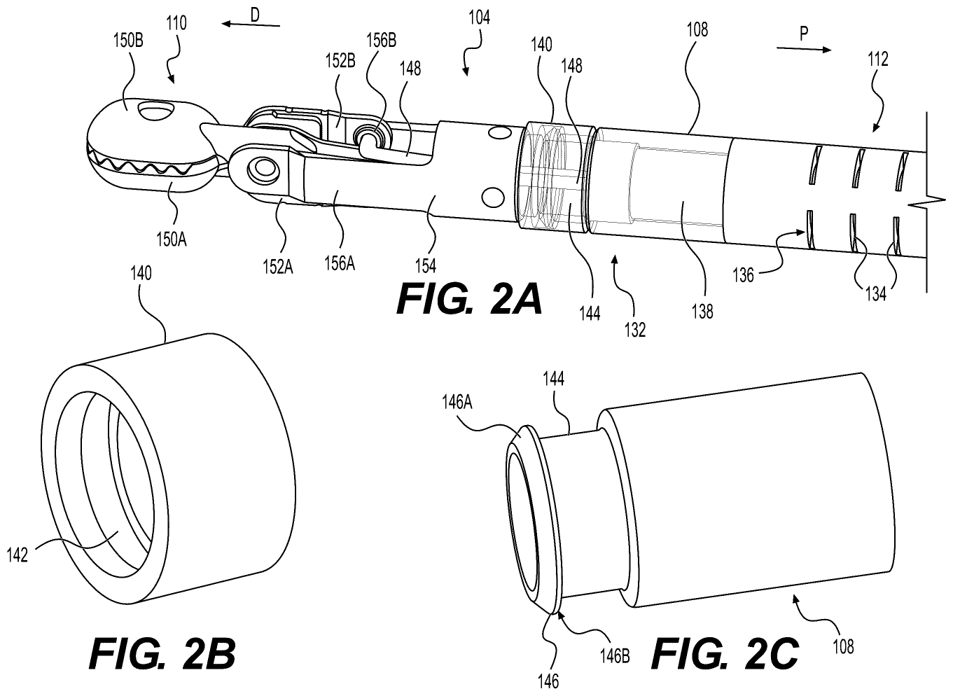

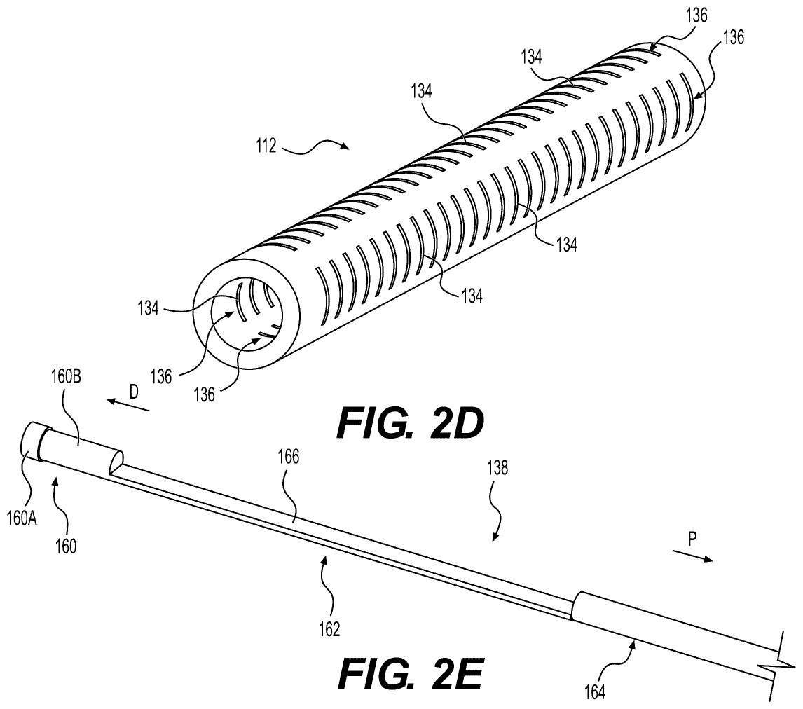

FIGS. 2A-2E illustrate various aspects of distal portion 104, for example, including the distal end of shaft 108, end effector 110, and deflectable portion 112. FIG. 2A illustrates internal connections between shaft 108 and end effector 110, and thus includes a distal portion of shaft 108 as being transparent. Nevertheless, shaft 108 may be at least partially opaque. Shaft 108 may include or otherwise radially surround a wire 138, for example, a pull wire or an actuation wire. For example, wire 138 may extend from handle 106 through shaft 108, and wire 138 may be movable (e.g., within shaft 108) via manipulation of one or more portions of handle 106 to control one or more aspects of distal portion 104 of medical device 100, for example, including end effector 110, deflectable portion 112, etc.

Coupling portion 132 may include a bushing or bush 140, which may be generally cylindrical and may also include an undercut 142 (FIG. 2B). Undercut 142 may be formed by a reduced radial thickness along an inner portion of bush 140, for example, extending circularly along the inner portion of bush 140. Additionally, a proximal portion of end effector 110 may be coupled to bush 140, for example, via a welding.

Moreover, shaft 108 may also include a distal extension 144, for example, including or being coupled to a flange portion 146, as shown particularly in FIG. 2C. As shown, distal extension 144 may be smaller than flange portion 146. For example, both distal extension 144 and flange portion 146 may be generally circular in a lateral cross-section, and distal extension 144 may include a smaller circumference than flange portion 146. Flange portion 146 may be positioned within undercut 142, for example, to rotatably couple shaft 108 to bush 140. For example, a distal portion 146A of flange portion 146 may include a beveled, slanted, or tapered surface, which may help for flange portion 146 to be positioned within undercut 142. Flange portion 146 includes a flat proximal portion 146B, and flange portion 146 may be positioned within undercut 142, helping to couple bush 140 and distal extension 144 (and thus bush 140 and shaft 108). In these aspects, a distal portion of shaft 108 may be rotatably coupled to bush 140, while the distal portion of shaft 108 is also fixed longitudinally. That is, bush 140 can rotate relative to shaft 108, but bush 140 does not move longitudinally relative to shaft 108.

Additionally, wire 138 may be coupled to end effector 110, for example, via one or more end effector wires 148. The one or more end effector wires 148 may extend radially within bush 140. In these aspects, rotation of wire 138 rotates end effector 110, and longitudinal movement of wire 138 controls or manipulates one or more aspects of end effector 110. For example, as discussed in detail below, wire 138 and end effector wire(s) 148 may be thick enough and/or fixedly coupled via one or more wires or other connections to convey rotational movement to end effector 110. Additionally, end effector 110 may be coupled to bush 140, which is rotatably couple to shaft 108. Moreover, longitudinal movement of wire 138 controls the movement of end effector wire(s) 148, which may actuate or otherwise manipulate end effector 110. In these aspects and as discussed below, movement of wire 138 may help to actuate or otherwise control end effector 110.

As shown in FIG. 2A, end effector 110 may include or otherwise be coupled to one or more end effector controls, for example, end effector wires 148. The one or more end effector wires 148 may be operably coupled to wire 138. For example, proximal end(s) of each end effector wire(s) 148 may be fixedly coupled to a distal end of wire 138 (e.g., either directly or indirectly). In these aspects, movement of wire 138 may control the movement of the one or more end effector wires 148, for example, to open and/or close or otherwise manipulate end effector 110. As shown, end effector 110 may be forceps, and may include a pair of jaws 150A, 150B. Movement of the one or more end effector wires 148 may open or close one or more of jaws 150A, 150B, for example, by pivoting jaws 150A, 150B such that distal ends of jaws 150A, 150B move away from each other (e.g., to open) or toward each other (e.g., to close). In some aspects, medical device 100 may include one end effector wire 148, for example, coupled to one of jaws 150A or 150B, such that movement of wire 138 moves the one end effector wire 148 to move one of jaws 150A or 150B away from the other of the jaws 150A or 150B. In other aspects, medical device 100 may include two end effector wires 148. In this example, one end effector wire 148 may be coupled to jaw 150A. Another end effector wire 148 may be coupled to jaw 150B, such that movement of wire 138 moves each of the two end effector wires 148 to move both of jaws 150A and 150B away from each other.

In some aspects, each of jaws 150A, 150B may include a proximal leg 152A, 152B. End effector 110 may include a proximal support 154, and proximal support 154 may include two distally extending posts 156A, 156B that form a clevis. Proximal legs 152A, 152B may be pivotably connected to respective posts 156A, 156B. In some aspects, one or more portions of end effector 110 (e.g., end effector wires 148) may be biased toward a configuration, for example, toward the closed configuration shown in FIG. 2A. Furthermore, a proximal end of end effector 110 (e.g., a proximal end of proximal support 154) may be coupled to bush 140, for example, welded or otherwise securely coupled (e.g., via a snap fit, a press fit, an adhesive, etc.).

FIG. 2D is a perspective view of deflectable portion 112. It is noted that deflectable portion 112 may be a portion of (e.g., integrally formed with) shaft 108, or deflectable portion 112 may be separately formed from shaft 108, and then coupled to shaft 108 and/or other components of medical device 100 during assembly. In either of these aspects, as mentioned above, deflectable portion 112 includes a plurality of openings 134. Openings 134 may be arranged circumferentially and longitudinally around deflectable portion 112. For example, deflectable portion 112 may include four longitudinally extending rows 136 of longitudinally spaced openings 134, with the rows 136 of openings 134 arranged circumferentially around deflectable portion 112. In other aspects, deflectable portion 112 may include fewer or more rows 136 of openings 134, for example, one row 136, two rows 136, three rows 136, five rows 136, etc. arranged circumferentially around deflectable portion 112.

FIG. 2E illustrates a distal portion of wire 138. As shown, the distal portion of wire 138 includes a distal section 160, an intermediate section 162, and a proximal section 164. Distal section 160 may include a stepped diameter portion 160A and a cylindrical distal portion 160B, with stepped diameter portion 160A including a larger cross-sectional diameter than cylindrical distal portion 160B. Additionally, intermediate section 162 includes a smaller lateral cross-section than distal section 160 and/or proximal section 164. Intermediate section 162 may include a semi-circular (or another partially circular) lateral cross-section. For example, intermediate section 162 may be formed by cutting away (e.g., an eccentric cut) or otherwise removing a portion of the distal portion of wire 138. In this aspect, intermediate section 162 may include a flat surface 166. As discussed below, flat surface 166 may at least partially align with and/or face one of the rows 136 of openings 134. Manipulation of wire 138 may bend intermediate section 162, for example, because stepped diameter portion 160A may be abutting a portion of shaft 108 (e.g., a distal end of deflectable portion 112) and because flat surface 166 is smaller or narrower than other portions of wire 138. Additionally, flat surface 166 may interact with one of the rows of openings 134 to help form an articulation joint and articulate a portion of distal portion 104 of medical device 100.

In some aspects, proximal section 164 may extend proximally to handle 106. For example, proximal section 164 may be coupled (i.e., directly or indirectly) to spool 118, such that movement of spool 118 controls an extension and/or retraction of proximal section 164. In these aspects, wire 138 may be moveable within shaft 108.

FIG. 3 is a partially cutaway view of proximal portion 102 of the medical device of FIG. 1, including handle 106. Handle 106 includes spool 118 that is movable within relative to main body 114, for example, within a portion of slot 120. Additionally, handle 106 includes spring or biasing element 122, for example, at least partially positioned within slot 120, for example, using the transition from wide portion 120A to narrow portion 120B as a distal stop surface 120C for biasing element 122. Alternatively, biasing element 122 may extend distal to distal top surface 120C to another portion of handle 106. Spool 118 may include an internal projection 170, for example, that extends within a portion of slot 120. In some aspects, internal projection 170 may be movable within slot 120, including wide portion 120A and narrow portion 120B, as spool 118 moves along slot 120. Internal projection 170 or another portion of spool 118 may be coupled to a proximal end of wire 138, for example, via a crimp 172 (e.g., a cylindrical crimp). A proximal end of biasing element 122 may be coupled to a distal end of projection 170. Alternatively, biasing element 122 may not be coupled to the distal end of projection 170, but may abut or otherwise interact with the distal end of the projection 170 as spool 118 is advanced distally within slot 120 Biasing element 122 may bias spool 118 proximally. For example, biasing element 122 may compress as spool 118 is moved distally, and biasing element 122 may urge spool 118 proximally (e.g., to the position shown in FIG. 3) once the distal force on spool 118 is removed. In some aspects, biasing element 122 may help to urge spool 118 proximally, and thus help to maintain end effector 110 in a closed configuration. The closed configuration may help to ensure that end effector 110 does not damage a working channel of an insertion device (e.g., an endoscope), damage tissue, etc. as medical device 100 is delivered to the treatment site.

Moreover, as shown in FIG. 3, a portion of proximal section 164 of wire 138 may be coupled to or otherwise interact with knob 124. For example, knob 124 may be formed of two halves that are coupled together to surround a portion of wire 138. Additionally, a tube, for example, a hypotube 174, may be attached or otherwise positioned around a proximal portion of wire 138. Hypotube 174 may include one or more flat outer surfaces, for example, including a square cross-section with four flat outer surface. In some aspects, hypotube 174 may extend proximally to spool 118, for example, through biasing element 122 and/or to internal projection 170. Knob 124 may surround wire 138 and hypotube 174, such that knob 124 engages with hypotube 174 to help rotate hypotube 174 and wire 138 as knob 124 rotates. Moreover, although not shown, handle 106 may include one or more braking or locking mechanisms. For example, an O-ring 176, gasket, or other mechanism may surround a portion of wire 138 and/or hypotube 174, and may help to provide a passive brake or frictional lock to help secure the position of wire 138 and spool 118 (and thus end effector 110) relative to handle 106. FIG. 3 illustrates O-ring 176 in a cross-sectional view at least partially surrounding hypotube 174 and wire 138. An exterior of O-ring 176 may interact with one or more internal portions of handle 106, for example, one or more internal portions of slot 120, to as spool 118 moves proximally or distally in slot 120.

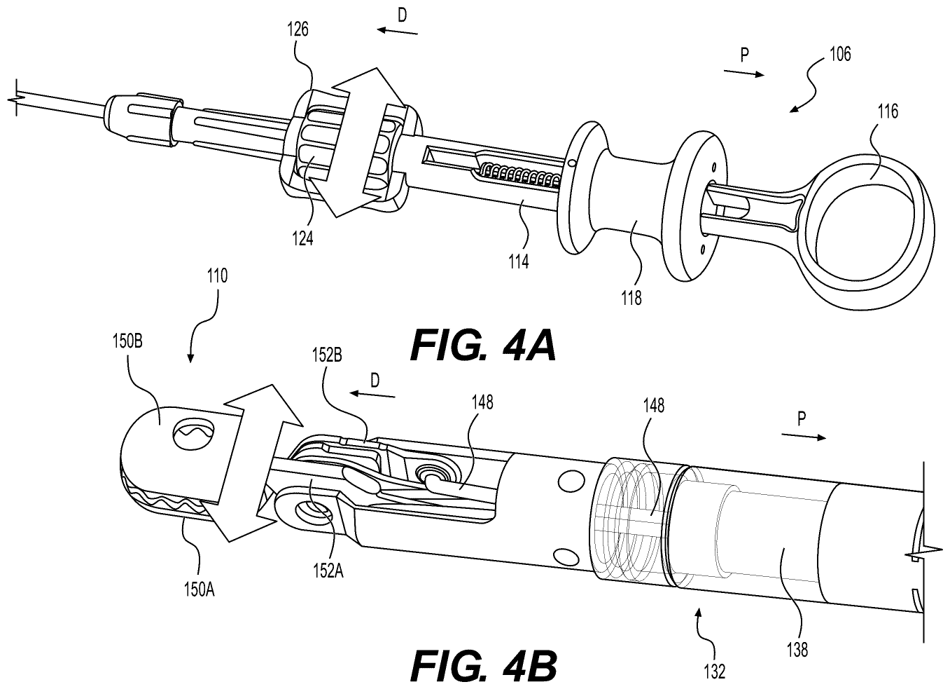

In these aspects and as shown in FIGS. 4A and 4B, rotation of knob 124 (e.g., clockwise or counterclockwise) may also rotate wire 138, and thus also end effector 110 (e.g., clockwise or counterclockwise about central a longitudinal axis of wire 138 and/or a central longitudinal axis of shaft 108), for example, via the connection via end effector wire(s) 148. For example, the user may rotate knob 124 (e.g., relative to cage 126 about a central longitudinal axis of knob 124) to rotate wire 138 (via hypotube 174) and end effector 110 to orient or otherwise position end effector 110 at the treatment site. In these aspects, the user may rotate knob 124 clockwise, which also rotates wire 138 and end effector 110 clockwise. Similarly, the user may rotate knob 124 counterclockwise, which also rotates wire 138 and end effector 110 counterclockwise. In these aspects, the rotation of knob 124, and thus of wire 138 and end effector 110, is independent of any rotation of shaft 108 and deflectable portion 112. Although not shown, knob 124 may include one or more internal springs or biasing elements, for example, to help bias knob 124 to one or more neutral or relaxed positions. In these aspects, knob 124 allows for end effector 110 to have a first degree of freedom (e.g., rotation in the clockwise direction or rotation in the counterclockwise direction).

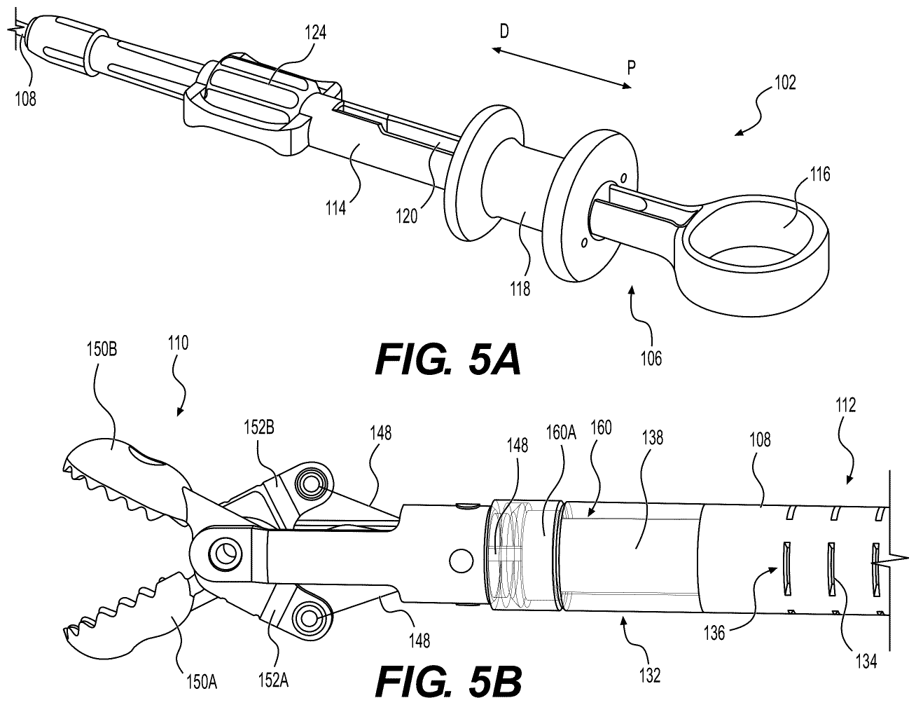

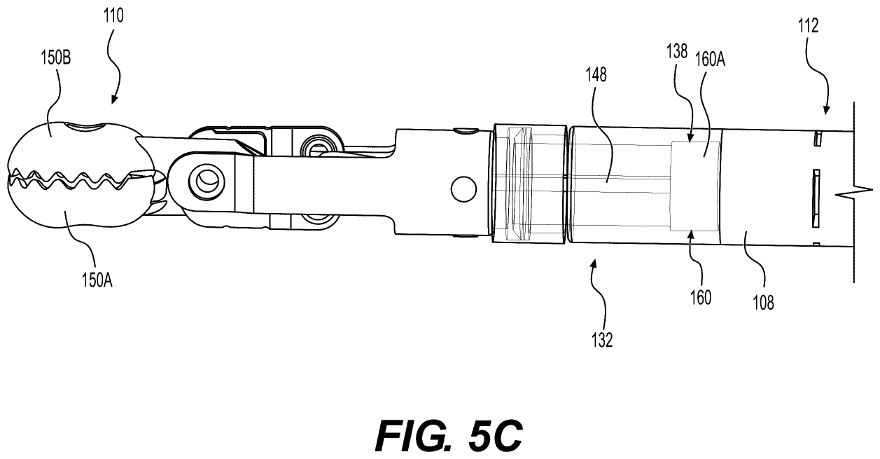

As shown in FIGS. 5A-5C, movement of spool 118 may control the actuation of end effector 110. For example, longitudinal movement of spool 118 in a first direction (e.g., in the distal direction) may open jaws 150A, 150B of end effector 110. As shown in FIG. 5B, stepped diameter portion 160A of distal section 160 of wire 138 may be positioned in a distal position, for example, adjacent to a proximal end of end effector 110. Additionally, longitudinal movement of spool 118 in a second, opposite direction (e.g., in the proximal direction) may close jaws 150A, 150B of end effector 110. As shown in FIG. 5C, stepped diameter portion 160A of distal section 160 of wire 138 may be positioned in a proximal position, for example, adjacent to deflectable portion 112. For example, stepped diameter portion 160A may mate with or otherwise abut a distal end of deflectable portion 112, helping to inhibit wire 138 from moving further proximally. Moreover, in these aspects, the movement of wire 138, and the resulting movement of end effector 110, is independent of any movement of shaft 108 and deflectable portion 112. As mentioned above, the movement of spool 118 may be biased by biasing element 122 (FIG. 3), for example, biased towards the closed configuration. In these aspects, movement of spool 118 allows for end effector 110 to have a second degree of freedom (e.g., opening and closing).

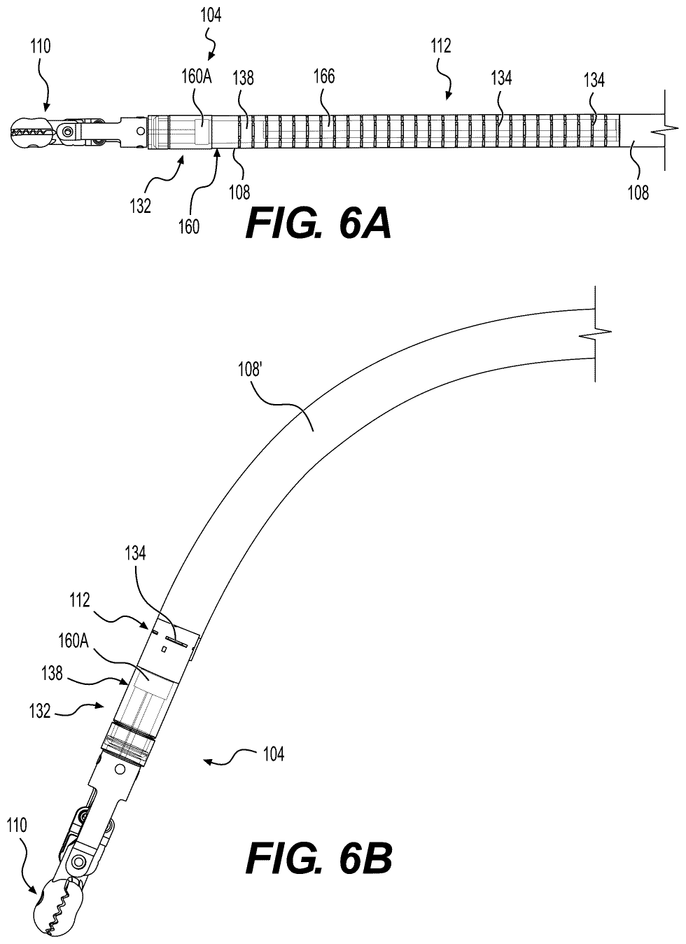

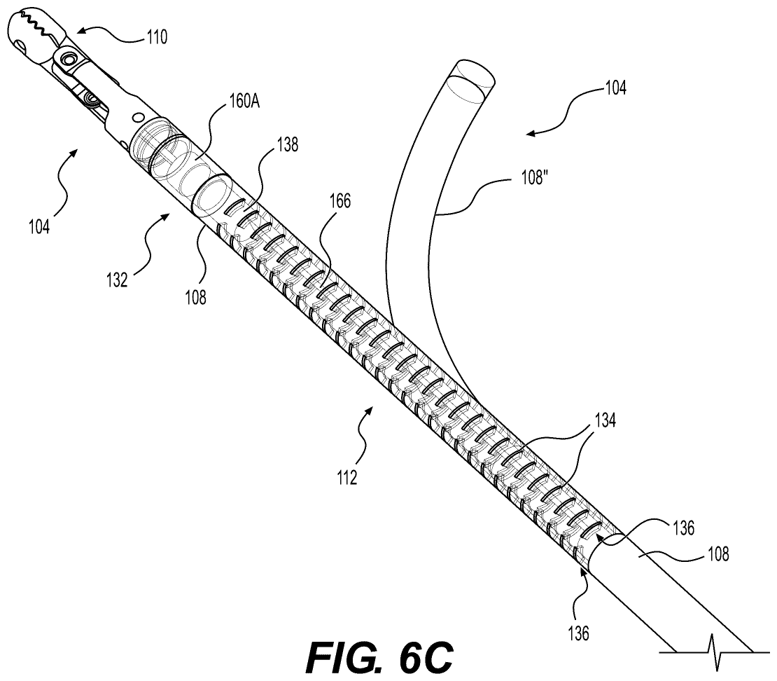

As shown in FIGS. 6A-6C, further or additional movement of spool 118 (FIG. 5A) may control the position (e.g., deflection) of distal portion 104, for example, by deflecting deflectable portion 112 to further control the position of end effector 110. As mentioned, spool 118 is coupled to a proximal end of wire 138. Proximal movement of spool 118 may move or urge wire 138 proximally. As mentioned, wire 138 includes stepped diameter portion 160A at distal section 160. Stepped diameter portion 160A of distal section 160 may abut a distal end of shaft 108 (e.g., the distal end of deflectable portion 112), and intermediate section 162 may overlap with at least a portion of deflectable portion 112. In these aspects, wire 138 may be retracted proximally and jaws 150A, 150B of end effector 110 may be closed. In these aspects, proximal movement of spool 118 causes deflectable portion 112 of shaft 108 to deflect, articulate, bend, or otherwise move, for example, to the position shown by shaft 108′ in FIG. 6C. For example, with stepped diameter portion 160A abutting the distal end of deflectable portion 112, wire 138 may act as a steering wire, for example, as wire 138 bends over narrower intermediate section 162 (e.g., due to flat surface 166). In these aspects, end effector 110 may be used to grasp tissue, and then closed (e.g., via biasing element 122. With the tissue secured within end effector 110, the user may manipulate (e.g., proximally retract) spool 118 to deflect, articulate, bend, or otherwise move shaft 108 and end effector 110, for example, to help lift or move the tissue.

Moreover, the position or orientation of wire 138 may affect the direction that shaft 108 (via deflectable portion 112) moves. For example, intermediate section 162 (including flat surface 166) may be positioned to be aligned with different rows 136 of openings 134 of deflectable portion 112. In these aspects, rotation of wire 138 to adjust the position of intermediate section 162 also rotates end effector 110, so the user may set or determine a deflection direction (e.g., a position or orientation of intermediate section 162 and flat surface 166) before grabbing or otherwise manipulating tissue with end effector 110. However, in some aspects, rotating end effector 110 when coupled to tissue may be desired or acceptable during the procedure. Next, proximal movement of spool 118 may urge wire 138 proximally. Because distal extension 144 of wire 138 is abutting a portion of shaft 108 (e.g., the distal end of deflectable portion 112) when jaws 150A, 150 of end effector 110 are closed, wire 138 may not translation proximally. Instead, wire 138 (e.g., intermediate section 162) bends in the direction that flat surface 166 is facing. In these aspects, flat surface 166 may be facing one row 136 of openings 134 of deflectable portion 112, such that the bending of wire 138 articulates, steers, or otherwise positions a distal portion of shaft 108 (e.g., deflectable portion 112), which also articulates, steers, or otherwise positions end effector 110.

Additionally, wire 138 may be rotated (e.g., via knob 124) such that flat surface 166 of intermediate section 162 aligns with (faces) another row 136 of openings 134. For example, as shown in FIG. 6B, with flat surface 166 facing downward, a proximal force on wire 138 helps to articulate, steer, or otherwise position shaft 108 in a downward direction (e.g., shaft 108′). Additionally, as shown in FIG. 6C, with flat surface 166 facing to the left, proximal force on wire 138 helps to articulate, steer, or otherwise position shaft 108 in a leftward direction (e.g., shaft 108″). Although not shown, wire 138, and thus flat surface 166, may be positioned to align with other rows 136 of openings 134, for example, to help articulate, steer, or otherwise position shaft 108 in an upward direction or in a rightward direction. In these aspects, wire 138 may be urged proximally (e.g., via spool 118) to help articulate, steer, or otherwise position shaft 108 (e.g., deflectable portion 112) and end effector 110 in a variety of directions, for example, in a plurality of known and/or predictable planes. If there are four rows 136 of openings 134 in deflectable portion 112, then shaft 108 and end effector 110 may be articulated, steered, or otherwise positioned in four directions (e.g., up, down, left, and right). A number of rows of openings 134 may correspond to a number of articulation directions. In some examples, handle 106 may include indicators to show a direction of rotation of flat surface 166/a direction that deflectable portion 112 will deflect upon actuation. Furthermore, the amount of proximal force or the degree to which spool 118 is proximally retracted may help to control the degree to which shaft 108 and end effector 110 are articulated, steered, or otherwise positioned.

In some aspects, distally urging wire 138 may also help to articulate, steer, or otherwise position shaft 108. For example, if medical device 100 does not include movable end effector, but instead includes a fixed end effector (e.g., a cytology brush), then distal movement or force on wire 138 may articulate, steer, or otherwise position shaft 108 in a direction opposite to the direction that flat surface 166 is facing. For example, spool 118 may be distally advanced such that distal extension 144 of wire 138 is abutting a distal end of shaft 108. Then, further distal movement of spool 118 may cause wire 138 (e.g., intermediate section 162) to bend in the opposite to the direction that flat surface 166 is facing. In these aspects, if flat surface 166 is facing one row 136 of openings 134 of deflectable portion 112 (e.g., the left side), then bending of wire 138 when urged distally causes wire 138 to bend in a direction opposite (e.g., to the right side) to the row 136 of openings 134 that flat surface 166 is facing. As mentioned above, the bending of wire 138 articulates, steers, or otherwise positions a distal portion of shaft 108 (e.g., deflectable portion 112), which also articulates, steers, or otherwise positions end effector 110.

Additionally, as mentioned, the proximal end of biasing element 122 may coupled to spool 118 (e.g., via internal projection 170 and/or crimp 172). In these aspects, biasing element 122 may bias spool 118, and thus wire 138, toward a neutral or relaxed position (e.g., as shown in FIG. 1). In these aspects, the manipulation of wire 138 may provide end effector 110 with third, fourth, fifth, and sixth degrees of freedom, all from the manipulation of a single wire (i.e., wire 138).

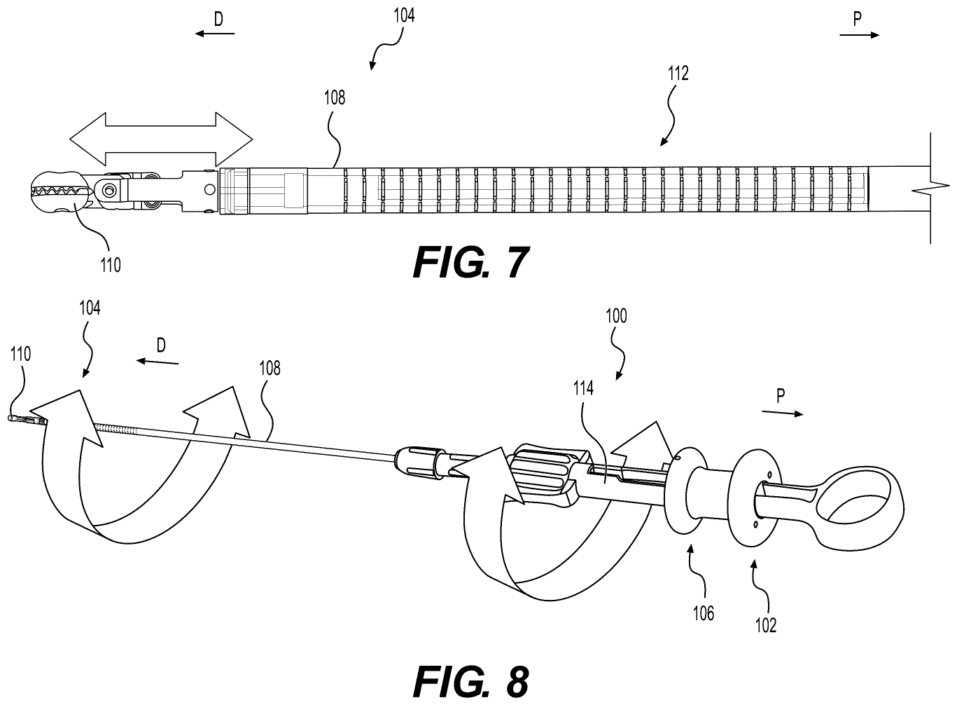

As shown in FIG. 7, distal portion 104, including shaft 108 and end effector 110, may be advanced distally or retracted proximally. For example, the entirety of medical device 100 (FIG. 1) may be advanced distally or retracted proximally, for example, by the user distally pushing, proximally pulling, or otherwise urging or manipulating handle 106 and/or shaft 108. In this aspect, medical device 100, including end effector 110, may include another (e.g., a seventh) degree of freedom.

As shown in FIG. 8, shaft 108 may be rotated, for example, clockwise and/or counterclockwise. For example, the entirety of medical device 100 (i.e., both proximal portion 102 and distal portion 104) may be rotated clockwise and/or rotated counterclockwise, for example, by the user rotating handle 106 (i.e., including main body 114) and/or shaft 108. In this aspect, medical device 100, including end effector 110, may include yet another (e.g., an eighth) degree of freedom.

Although not shown, in some aspects, a distal portion of shaft 108 may include a pre-shaped section. For example, a distal portion of shaft 108 may include a bent or arc shape. In this aspect, shaft 108 may be delivered to the treatment site via a sheath. The sheath may be more rigid than the distal portion of shaft 108, for example, such that the distal portion of shaft 108 is straight when positioned within the sheath. Then, once shaft 108 is positioned at the treatment site, the sheath may be proximally retracted, such that the distal portion of shaft 108 is exposed. Alternatively, shaft 108 may be delivered to the treatment site via an insertion device (e.g., through a working channel of an endoscope, ureteroscope, catheter, etc.), and extending a portion of shaft 108 distally of the insertion device may expose a portion of shaft 108. In these aspects, the exposed portion of shaft 108 may return to the bent or arc shape based on shaft 108 including a pre-shaped section, for example, articulating, steering, or otherwise positioning shaft 108. In these aspects, wire 138 may still be used to manipulate end effector 110, as discussed above. Nevertheless, wire 138 may not include intermediate section 162 with flat surface 166, as shaft 108 may be movable on its own based on the pre-shaped section and the movement of the sheath.

Various aspects of medical device 100 may allow for an end effector (i.e., end effector 110) to be manipulated through at least six degrees of freedom with a by manipulation of a single wire (i.e., wire 138). These degrees of freedom are independent of the movement of the medical device as a whole, for example, as discussed above with respect to FIGS. 7 and 8. As mentioned, rotation of knob 124 allows the user to rotate end effector 110 clockwise or counterclockwise about a longitudinal axis of medical device 100. Additionally, longitudinal manipulation of spool 118 (e.g., distal of the neutral position) allows the user to actuate (e.g., open and close) end effector 110. Moreover, longitudinal manipulation of spool 118 (e.g., proximal of the neutral position) allows the user to deflect, articulate, or otherwise move a distal end of shaft 108, and thus also deflect, articulate, or otherwise move end effector 110, for example, in four different directions (e.g., leftward, rightward, upward, and downward). As mentioned, the direction of the deflection, articulation, or movement of shaft 108 and end effector 110 may be controlled based on the orientation of wire 138, that is, based on the orientation of flat surface 166 relative to deflectable portion 112 and its rows 136 of openings 134. The end effector 110 may also be moved along with the entirety of medical device 100 (e.g., as shown and discussed with respect to FIGS. 7 and 8). The positioning, actuation, repositioning, deflection, etc. may be performed as many times as necessary during a medical procedure. Additionally, as mentioned above, the same wire (e.g., wire 138) may be manipulated to position end effector 110, open or close end effector 110, and also deflect, articulate, move, or otherwise manipulate end effector 110, for example, to locate, grasp, and lift or otherwise reposition the tissue (e.g., in an electrosurgical dissection procedure). Because only a single wire 138 is utilized, wire 138 may be sufficiently thick so as to be robust and avoid breakage, degradation, or other harmful side effects of using a thinner control wire. Furthermore, a diameter of shaft 108 may be reduced because only one wire 138 is used.

Furthermore, the various components of handle 106 (e.g., spool 118, knob 124, etc.) may be operated by a single user, for example, with one or more hands (e.g., with a user's thumb positioned in ring 116). Handle 106 may provide an ergonomic design, and the controls on handle 106 may be controls that a user may already be familiar with (e.g., extending or retracting a spool, rotating a knob, etc.). Additionally, the single wire (i.e., wire 138) may allow for shaft 108 to be smaller (e.g., include a smaller cross-sectional profile), which may allow for shaft 108 to be delivered through a smaller working channel or otherwise be delivered to smaller lumens or portions of a subject's body. For example, shaft 108 may include a cross-sectional diameter or profile of approximately 2.2 mm, and shaft 108 may be delivered through a working channel with a cross-sectional diameter or profile of approximately 2.8 mm.

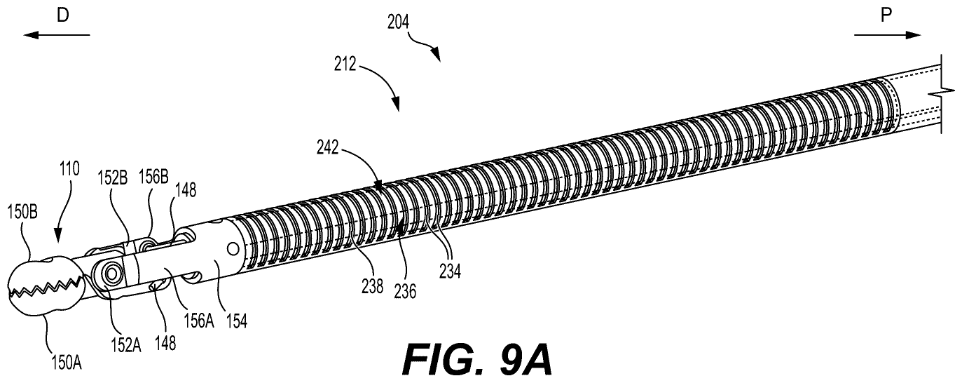

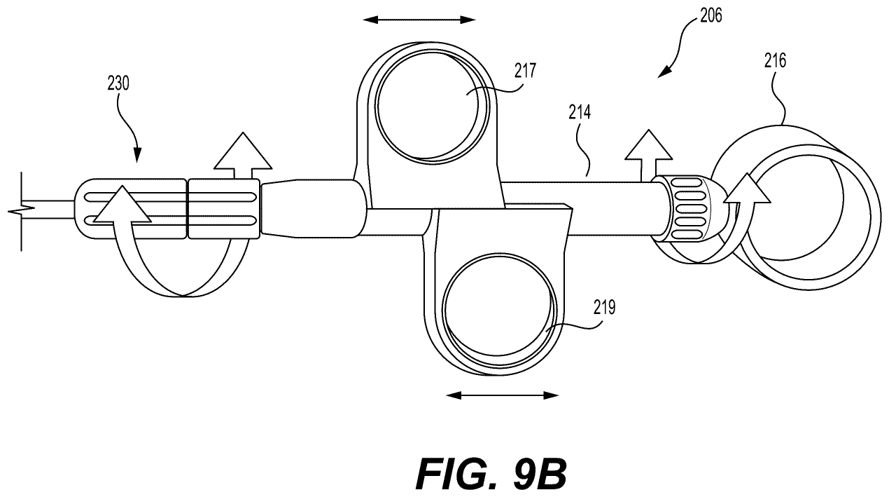

FIGS. 9A-13B illustrate aspects of an additional exemplary distal portion of a medical device 200, for example, similar to the medical device of FIG. 1. The aspects shown in FIGS. 9A-13B may include two independent pull wires for independent articulation and steering functionality. Distribution of articulation and steering functionality between two independent wires may help to improve ergonomics for an operator. Ergonomics may be improved, for example, by reducing the amount of force needed to actuate the two wires and by increasing the number of jaw and end effector articulation configurations available to an operator.

FIG. 9B illustrates a proximal portion of the medical device depicted in FIG. 9A. In some aspects, the two wires (e.g., a first wire 238 and a second wire 242) may be each be operably connected to a different actuator of handle 206. For example, wire 238 may be operably connected to an actuator 217, and wire 242 may be operably connected to an actuator 219. Actuator 217 and actuator 219 may each be a same type of actuator (e.g., spools, finger rings, etc.), though this is only exemplary and other known actuator configurations are within the scope of the disclosure. Handle 206 includes a main body 214, for example, including a ring 216 (e.g., a thumb ring), for example, at a proximal end of main body 114. A user may rotate ring 216 to rotate main body 214, a shaft 208, wire 238, end cap 230, wire 242, and an end effector 110, which may all rotate together. Handle 206 may also include an end cap 230, for example, at a distal end of handle 206. End cap 230 may surround a proximal end of shaft 208. End cap 230 may help to couple shaft 208 to handle 206. In some aspects, end cap 230 may help to form a strain relief portion of medical device 100.

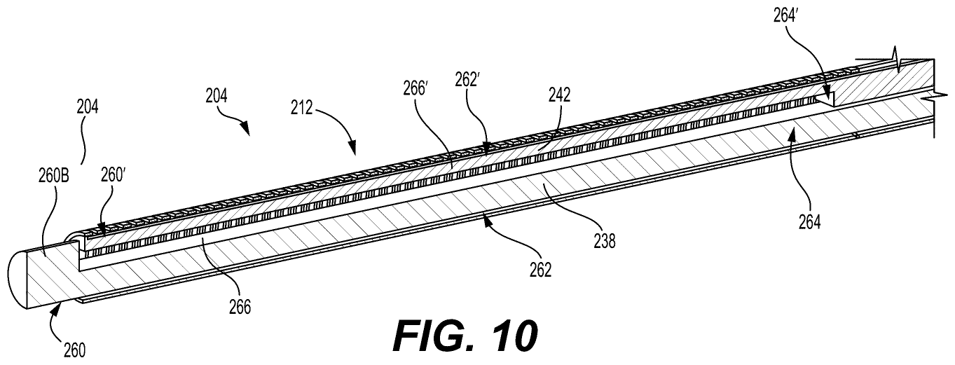

FIGS. 9A-10 illustrate various aspects of a distal portion 204 of shaft 208, for example, including the distal end of shaft 208, end effector 110, and a deflectable portion 212. FIG. 9A is a perspective view of distal portion 204, and FIG. 10 is a longitudinal cross-sectional view of distal portion 204. FIG. 10 illustrates internal connections between shaft 208 and end effector 110. Shaft 208 may include or otherwise radially surround first wire 238 and second wire 242, for example, each of which may be a pull wire and/or an actuation wire. For example, first wire 238 (and second wire 242) may extend from handle 206 through shaft 208, and first wire 238 (and second wire 242) may be movable (e.g., within shaft 208) via manipulation of one or more portions of handle 206 to control one or more aspects of distal portion 204 of medical device 200, for example, including end effector 110, deflectable portion 212, etc.

Referring now to FIG. 9A, shaft 208 may include deflectable portion 212. Deflectable portion 212 may include a tube with a plurality of grooves, slits, or openings 234, for example, extending around respective portions of a circumference of deflectable portion 212. Shaft 208 may be configured to accommodate first wire 238 (used for articulation of jaws 150A and 150B) and second wire 242 (used for articulation of deflectable portion 212).

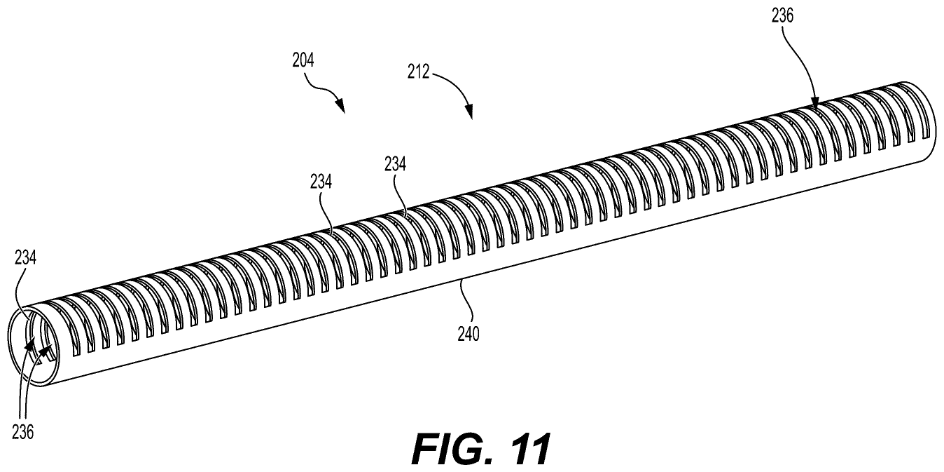

Deflectable portion 212 may include a plurality of openings 234 that are circumferentially positioned on only a portion (circumferentially) of deflectable portion 212. Referring to FIG. 11, each opening 234 may extend around approximately 180 degrees to approximately 330 degrees, for example, approximately 270 degrees of deflectable portion 212 of shaft 208. In contrast to openings 134 of deflectable portion 112 illustrated in FIG. 2D, openings 234 or deflectable portion 212 may span a relatively larger circumferential portion of shaft 208 than a non-perforated (e.g., without openings 234) portion 240 of shaft 208. For example, non-perforated portion 240 may span approximately 90 degrees and may more generally be defined by the portion (circumferentially) of shaft 208 without openings 234. Openings 234 may form a single longitudinally extending row 236. In some aspects, however, openings 234 may form more than one row 236. For example, two rows 236 may be formed by openings 234 each occupying approximately 135 degrees of deflectable portion 212.

As shown in FIG. 11, each opening 234 in row 236 is spaced longitudinally from an adjacent opening 234 in row 236. It will be noted, however, that the size and spacing (longitudinal spacing, longitudinal width, circumferential spacing, circumferential length, etc.) of openings 234 and row 236 may vary. In these aspects, openings 234 may be formed by laser cutting or otherwise cutting, for example, forming a laser cut pattern including row 236 of openings 234. Additionally, although not shown, shaft 208 may include one or more outer layers, for example, radially surrounding deflectable portion 212. In these aspects, the one or more outer layers may be flexible, such that movement of deflectable portion 212 (e.g., via second wire 242) moves shaft 208. Non-perforated portion 240 may be more resistant to bending (e.g. is stiffer, more rigid, and/or less flexible) compared to the circumferential portion of shaft 208 occupied by row 236 and openings 234. It is noted that various other ratios between the amount of the circumference of deflectable portion 212 occupied by non-perforated portion 240 and row 236 (e.g., openings 234) are possible, such as 1:1 (e.g., 180 degrees occupied by each of non-perforated portion 240 and row 236).

FIG. 9A shows end effector 110, jaws 150A and 150B, legs 152A and 152B, distally extending posts 156A and 156B, end effector wire(s) 148, and proximal support 154. End effector wires 148 may be connected to first wire 238 (shown in greater detail in FIG. 10). End effector 110 may be operably coupled to a distal portion 204 of shaft 208, including deflectable portion 212. Longitudinal movement of first wire 238 may move end effector wires 148, which may cause jaws 150A and 150B of end effector 110 to open and close. In these aspects and as discussed below, movement of first wire 238 may help to actuate or otherwise control end effector 110.

As mentioned above, FIG. 10 illustrates a cross-sectional view of shaft 208, first wire 238, and second wire 242. As shown, the distal portion of first wire 238 may include a distal section 260, an intermediate section 262, and a proximal section 264. Second wire 242 may include a distal section 260′, an intermediate section 262′, and a proximal section 264′.

With respect to first wire 238, distal section 260 may include a cylindrical distal section 260B, with cylindrical distal section 260B including a larger cross-sectional diameter than a flat surface 266 of an intermediate section 262 and/or proximal section 264. Intermediate section 262 may include a semi-circular (or another partially circular) lateral cross-section, forming a D-shaped cross-sectional shape with flat surface 266. For example, intermediate section 262 may be formed by cutting away (e.g., an eccentric cut) or otherwise removing a portion of the distal portion of first wire 238. Cylindrical distal section 260B may be connected to end effector wires 148 such that longitudinal movement of first wire 238 applies longitudinal force to end effector wires 148, which may help to open and close end effector 110.

With respect to second wire 242, intermediate section 262′ may include a semi-circular (or another partially circular) lateral cross-section, forming a D-shaped cross-sectional shape with a flat surface 266′. For example, intermediate section 262′ may be formed by cutting away (e.g., an eccentric cut) or otherwise removing a portion of the distal portion of second wire 242. In this aspect, intermediate section 262′ may include flat surface 266′. As discussed below, flat surface 266′ may at least partially align with and/or face a portion of row 236 of openings 234. Manipulation of second wire 242 may bend intermediate section 262′, for example, because distal section 260′ may be abutting a portion of shaft 208 (e.g., a distal end of deflectable portion 212) and because flat surface 266′ is smaller or narrower than other portions of second wire 242. Additionally, flat surface 266′ may interact with openings 234 to help form an articulation joint and articulate a portion of distal portion 204 of medical device 100. In some aspects, second wire 242 may be attached or otherwise be operably connected to shaft 208 (e.g., deflectable portion 212). Furthermore, second wire 242 (and first wire 238) may be rotatable. As discussed above, the direction that flat surface 266′ is facing during the manipulation of second wire 242 (e.g., proximal or distal movement or urging) may help to control the direction that shaft 208 (e.g., deflectable portion 212) bends. Moreover, shaft 208 may be rotatable (e.g., with handle 206) to further control the direction that shaft 208 (e.g., deflectable portion 212) bends, as discussed above with respect to medical device 100.

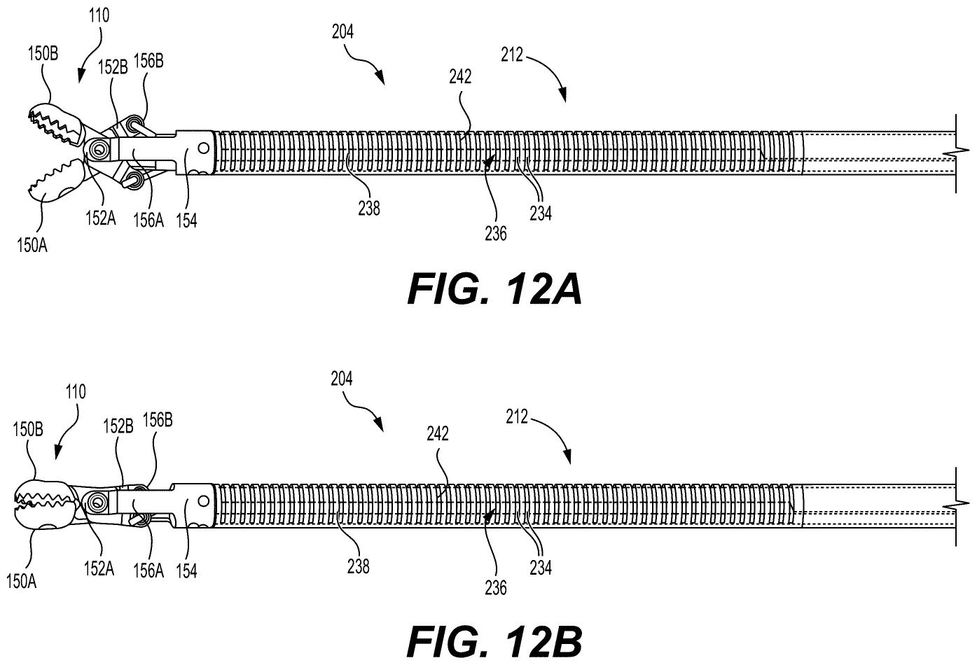

FIG. 12A-12B show articulation or movement of end effector 110 via movement of first wire 238. As shown in FIG. 12A, end effector 110 may include or otherwise be coupled to one or more end effector controls, for example, end effector wires 148. The one or more end effector wires 148 may be operably coupled to first wire 238. For example, proximal end(s) of each end effector wire(s) 148 may be fixedly coupled to distal section 260′ of first wire 238 (e.g., either directly or indirectly). In these aspects, movement of first wire 238 may control the movement of the one or more end effector wires 148, for example, to open and/or close or otherwise manipulate end effector 110. As shown, end effector 110 may be forceps, and may include a pair of jaws 150A, 150B. Movement of the one or more end effector wires 148 may open or close one or more of jaws 150A, 150B, for example, by pivoting jaws 150A, 150B such that distal ends of jaws 150A, 150B move away from each other (e.g., to open) or toward each other (e.g., to close). In some aspects, medical device 200 may include one end effector wire 148, for example, coupled to one of jaws 150A or 150B, such that movement of first wire 238 moves the one end effector wire 148 to move one of jaws 150A or 150B away from the other of the jaws 150A or 150B. In other aspects, medical device 200 may include two end effector wires 148. In this example, one end effector wire 148 may be coupled to jaw 150A. Another end effector wire 148 may be coupled to jaw 150B, such that movement of first wire 238 moves each of the two end effector wires 148 to move both of jaws 150A and 150B away from each other. End effector 110 may be opened by pushing first wire 238 proximally (shown in FIG. 12A) and end effector 110 may be closed by pulling first wire 238 proximally (show in FIG. 12B).

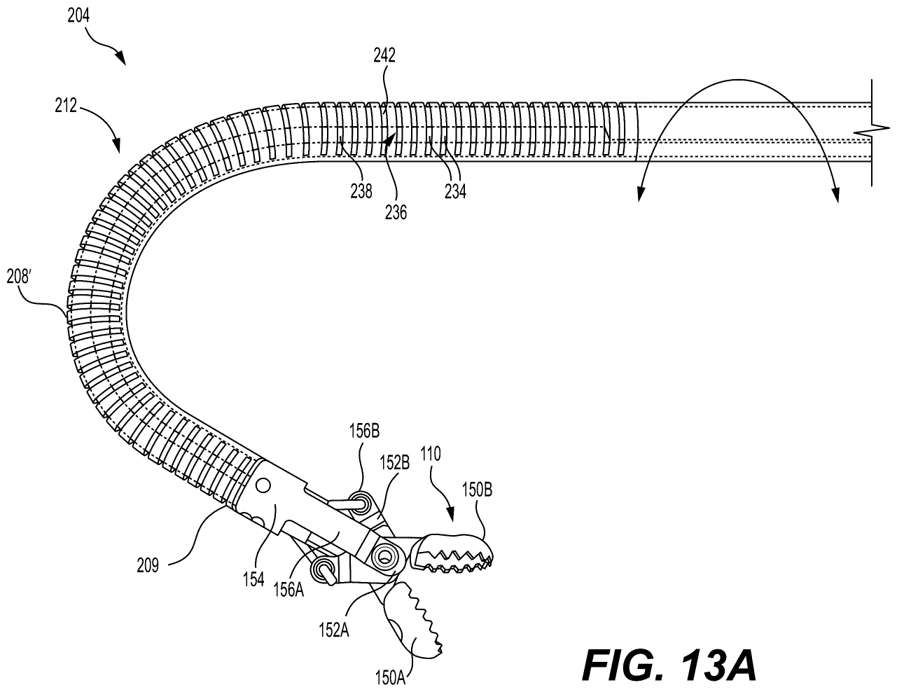

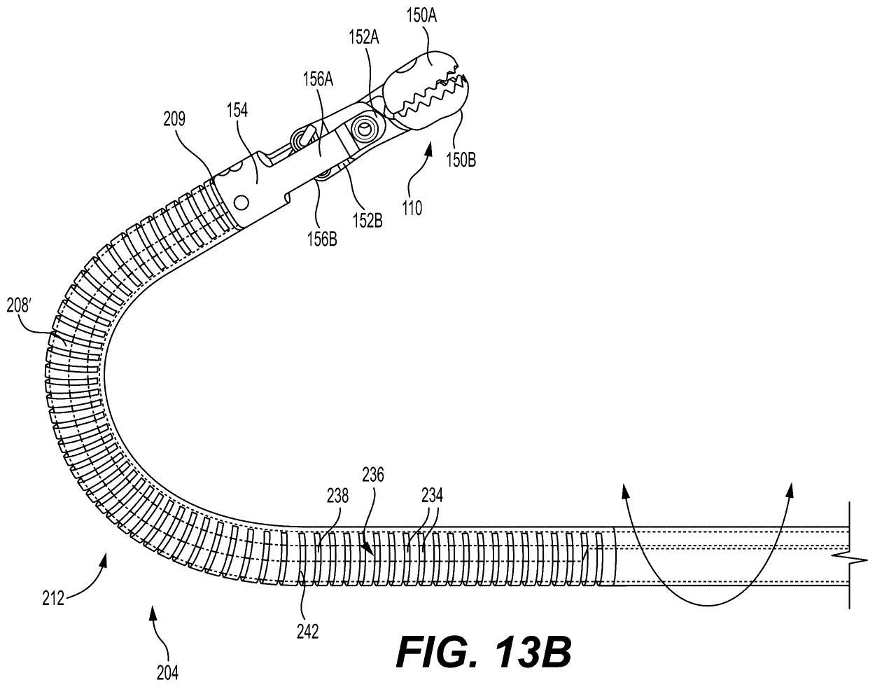

FIG. 13A-13B show deflection, steering, or movement of deflectable portion 212 via movement of second wire 242. As shown in FIGS. 13A-13B, further or additional movement of second wire 242 (shown in FIG. 10) may control the position (e.g., deflection) of distal portion 204, for example, by deflecting deflectable portion 212 to further control the position of end effector 110. As mentioned, second wire 242 is coupled to a distal end 209 of shaft 208 via distal section 260′. Proximal movement of second wire 242 may move or urge second wire 242 proximally. As mentioned, second wire 242 (e.g., distal section 260B) may abut distal end 209 of shaft 208 (e.g., the distal end of deflectable portion 212), and intermediate section 262 may overlap with at least a portion of deflectable portion 212. Proximal movement of second wire 242 causes deflectable portion 212 of shaft 108 to deflect, articulate, bend, or otherwise move, for example, to the position shown by shaft 208′ in FIG. 13A. For example, with second wire 242 abutting the distal end of deflectable portion 212, second wire 242 may act as a steering wire, for example, as second wire 242 bends over narrower intermediate section 262′ (e.g., due to flat surface 266). The user may manipulate (e.g., proximally retract) second wire 242 to deflect, articulate, steer, bend, or otherwise move shaft 208 and end effector 110. Another example of articulation of shaft 208′ is shown in FIG. 13B.

In the aspects illustrated in FIGS. 9-13, an operator may rotate shaft 208 to rotate both end effector 110 and intermediate section 262′ (e.g., flat surface 266′). Moreover, the position or orientation of second wire 242 may affect the direction that shaft 208 (via deflectable portion 212) deflects, articulates, steers, bends or otherwise moves. For example, intermediate section 262′ (including flat surface 266′) may be positioned to be aligned various portions of openings 234 of deflectable portion 212. As an example, an operator may rotate shaft 208 such that intermediate section 262′ may face the 6 o'clock position, such that distal movement of second wire 242 articulates deflectable portion 212 towards (and in some instances, past) the 6 o'clock position (shown in FIG. 13A). Similarly, an operator may rotate shaft 208, such that intermediate section 262′ may face the 12 o'clock position such that distal movement of second wire 242 articulates deflectable portion 212 towards (and in some instances, past) the 12 o'clock position (shown in FIG. 13B). It will be understood that rotation of shaft 208 rotates end effector 110, first wire 238, and second wire 242. Thus, rotation of shaft 208 determines a deflection direction (e.g., a position or orientation of intermediate section 262′ and flat surface 266′) before grabbing or otherwise manipulating tissue with end effector 110 (via movement of first wire 238).

Next, proximal movement of second wire 242 may urge second wire 242 proximally. Second wire 242 (e.g., intermediate section 262′) bends in the direction that flat surface 266 is facing. In these aspects, flat surface 266′ may be facing row 236 of openings 234 of deflectable portion 212, such that the bending of second wire 242 articulates, steers, or otherwise positions a distal portion of shaft 208 (e.g., deflectable portion 212), which also articulates, steers, or otherwise positions end effector 110.

In some examples, handle 206 may include indicators to show a direction of rotation of flat surface 266′ or a direction that deflectable portion 212 will deflect upon actuation of second wire 242. Furthermore, the amount of proximal force or the degree to which second wire 242 is proximally retracted or distally extended may help to control the degree to which shaft 208 and end effector 110 are articulated, steered, or otherwise positioned.

It will be apparent that actuation of end effector 110 and articulation of deflectable portion 212 may be independent of one another. For example, a user may actuate (e.g., open) end effector 110 by pushing first wire 238 distally, and may simultaneously articulate deflectable portion 212 in a first direction by pushing second wire 242 distally (shown in FIG. 13A). In another example, a user may actuate (e.g., close) end effector by pulling first wire 238 proximally, and may simultaneously articulate deflectable portion 212 in a second direction by pulling second wire 242 proximally (shown in FIG. 13B).

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a handle, wherein the handle includes a rotatable knob and a longitudinally movable spool;
a shaft extending from a distal end of the handle;
an end effector coupled to a distal end of the shaft; and
a wire extending from the handle, through the shaft, and to the end effector,
wherein rotation of the knob rotates the wire independent of the shaft to rotate the end effector,
wherein distal movement of the spool moves the wire distally independent of the shaft and transitions the end effector between at least a first configuration and a second configuration, and
wherein proximal movement of the spool urges the wire proximally, and wherein urging the wire proximally deflects a deflectable portion of the shaft in one or more directions.

2. The medical device of claim 1, wherein the shaft includes a deflectable portion that includes a plurality of openings arranged in one or more longitudinally extending rows.

3. The medical device of claim 2, wherein the deflectable portion includes four longitudinally extending rows of openings that are spaced circumferentially around the deflectable portion.

4. The medical device of claim 3, wherein a portion of the wire includes an intermediate section including a flat surface.

5. The medical device of claim 4 wherein the flat surface at least partially overlaps with at least one of the rows of openings.

6. The medical device of claim 5, wherein the flat surface is configured to overlap with a respective row of the four longitudinally extending rows based on a rotational position of the wire in order to deflect the deflectable portion in four directions.

7. The medical device of claim 1, further comprising a biasing element positioned within a portion of the handle to bias the distal movement of the spool.

8. The medical device of claim 1, further comprising a tube surrounding a proximal portion of the wire, wherein the tube includes at least one flat outer surface to interact with an internal portion of the knob.

9. The medical device of claim 1, wherein the end effector includes two pivotable jaws that are movable between a closed configuration and an open configuration.

10. The medical device of claim 9, wherein the two pivotable jaws are coupled to a distal end of the wire via respective end effector wires.

11. The medical device of claim 10, wherein a proximal end of the end effector is coupled to a bushing, and wherein the bushing is rotatably coupled to a distal end of the shaft.

12. The medical device of claim 1, wherein a distal end of the wire includes a stepped diameter portion that is larger than a proximal portion of the wire.

13. The medical device of claim 1, wherein the handle includes a handle body with a slot that extends longitudinally through a portion of the handle body, wherein a portion of the spool is movable within the slot to control the longitudinal movement of the wire, and wherein the slot includes a narrow distal portion and a wide proximal portion.

14. The medical device of claim 1, wherein the handle includes one or more arms that partially surround the knob.

15. The medical device of claim 1, wherein a proximal end of the handle includes a ring.

16. A medical device, comprising:
a handle, wherein the handle includes a rotatable knob and a longitudinally movable spool;
a shaft extending from a distal end of the handle;
an end effector rotatably coupled to a distal end of the shaft; and
a wire extending from the handle, through the shaft, and to the end effector, wherein a distal portion of the wire includes a distal extension and an intermediate section including a flat surface,
wherein rotation of the knob rotates the wire independent of the shaft to rotate the end effector, and
wherein distal movement of the spool moves the wire distally independent of the shaft and transitions the end effector between at least a first configuration and a second configuration.

17. The medical device of claim 16, wherein the shaft includes a deflectable portion that includes four longitudinally extending rows of openings that are spaced circumferentially around the deflectable portion, wherein the flat surface at least partially overlaps with at least one of the rows of openings, such that proximal movement of the spool urges the wire proximally, and wherein urging the wire proximally deflects the deflectable portion in one or more directions.

18. The medical device of claim 17, further comprising a tube surrounding a proximal portion of the wire, wherein the tube includes at least one flat outer surface to interact with an internal portion of the knob.

19. A medical device, comprising:
a handle, the handle including:
a first longitudinally movable actuator; and
a second longitudinally movable actuator;
a shaft extending from a distal end of the handle, the shaft including a deflectable portion that includes a plurality of openings arranged in a longitudinally extending row;
an end effector coupled to a distal end of the shaft;
a first wire extending from the handle, through the shaft, and to the end effector; and
a second wire extending from the handle, through the shaft, and to the distal end of the shaft,
wherein rotation of the shaft rotates the end effector, the first wire, and the second wire,
wherein distal movement of the first actuator moves the first wire distally independent of the shaft and the second wire, and transitions the end effector between at least a first configuration and a second configuration, and
wherein proximal movement of the second actuator urges the second wire proximally independent of the first wire, and wherein urging the second wire proximally deflects the deflectable portion of the shaft in one or more directions.

20. The medical device of claim 19, wherein the first wire includes a cylindrical distal section configured to couple to the end effector, wherein the second wire includes an intermediate section including a flat surface, wherein the flat surface at least partially overlaps with the longitudinally extending row, wherein the handle includes a third actuator, the third actuator rotatably coupled to the second wire, wherein rotation of the second wire via the third actuator is independent of the shaft and the first wire.
